(19) United States
(12) Patent Application Publication
(10) Pub. No.: US 2011/0189048 A1
(43) Pub. Date: Aug. 4, 2011

(54) MODULAR DIALYSIS SYSTEM

(76) Inventors: James R. Curtis, Portland, OR (US); Michael Baker, Harbor, WA (US); Dalibor Jan Smejtek, Portland, OR (US)

(21) Appl. No.: 12/960,373

(22) Filed: Dec. 3, 2010

Related U.S. Application Data
(60) Provisional application No. 61/267,046, filed on Dec. 5, 2009.

(51) Int. Cl. B01D 61/30; A61L 2/18; A61L 2/24

(52) U.S. Cl. 422/3; 210/85; 210/96.2; 422/292

(57) ABSTRACT

A dialysis system includes a plurality of modular components. The modular components can be coupled to one another in various configurations, wherein each configuration is optimized for use in a particular environment, such as in a home environment or a travel environment or a dialysis center.

### REFERENCE TO PRIORITY DOCUMENT

This application claims priority of co-pending U.S. Provisional Patent Application Ser. No. 61/267,046, entitled “Dialysis System Sanitation” and filed Dec. 5, 2009. Priority of the aforementioned filing date is hereby claimed and the disclosure of the Provisional Patent Application is hereby incorporated by reference in its entirety.

### BACKGROUND

There are, at present, hundreds of thousands of patients in the United States with end-stage renal disease. Most of those require dialysis to survive. United States Renal Data System projects the number of patients in the U.S. on dialysis will climb past 600,000 by 2012.

Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Given the demanding nature of in-center dialysis, many patients have turned to home dialysis as an option. Home dialysis provides the patient with ability to perform dialysis in the comfort of his or her home. Home dialysis further provides the patient with scheduling flexibility as it permits the patient to choose treatment times to fit other activities, such as going to work or caring for a family member. However, current home dialysis systems can be large and heavy, making it difficult for a user to transport the home dialysis system for use in environments outside the home.

### SUMMARY

In view of the foregoing, there is a need for improved dialysis system for use in a patient's home. Disclosed is a home dialysis system that includes a plurality of modular components. The modular components can be coupled to one another in various configurations, wherein each configuration is optimized for use in a particular environment, such as in a home environment or a travel environment or a dialysis center.

In one aspect, there is disclosed a modular dialysis system, comprising: a plurality of modules adapted to be operatively removably coupled together to collectively form a dialysis system capable of performing a dialysis procedure on a patient, the modules including: a user interface module comprising at least one user input element and at least one display element; a water treatment module comprising water treatment components configured to treat water for use in the dialysis procedure; and a dialysis module comprising components configured to perform dialysis.

Other features and advantages should be apparent from the following description of various embodiments, which illustrate, by way of example, the principles of the disclosed devices and methods.

### BRIEF DESCRIPTION OF THE DRAWINGS

FIGS. 1A and 1B show a dialysis system that is configured to perform dialysis on a patient.

FIG. 2 shows a high level, schematic view of an exemplary dialysis system.

FIG. 3 shows exemplary embodiments of a first module coupled to a second module of the dialysis system.

FIGS. 4-6 show the modules in various states of transport.

FIG. 7 shows an exemplary user interface module of the dialysis system.

FIG. 8 shows a coupling port for the user interface module.

FIGS. 9-11 show the user interface module in various states of use with the dialysis system.

FIG. 12 shows an exemplary embodiment of a first module of the dialysis system.

FIGS. 13A and 13B show access states of the first module.

FIG. 14 shows a plan view of an exemplary cartridge module.

FIGS. 15-17 show an exemplary method of coupling the cartridge module to the dialysis system.

FIGS. 18-20 show an exemplary method of coupling a dialysate bag to the dialysis system.

FIG. 21 shows the dialysis system in use.

FIG. 22 shows a schematic diagram of another embodiment of a modular dialysis system.

FIGS. 23 and 24 show an exemplary flow pathway that forms a mixing chamber for mixing ultra-pure water with an acid concentrate

### DETAILED DESCRIPTION

In order to promote an understanding of the principals of the disclosure, reference is made to the drawings and the embodiments illustrated therein. Nevertheless, it will be understood that the drawings are illustrative and no limitation of the scope of the disclosure is thereby intended. Any such alterations and further modifications in the illustrated embodiments, and any such further applications of the principles of the disclosure as illustrated herein are contemplated as would normally occur to one of ordinary skill in the art.

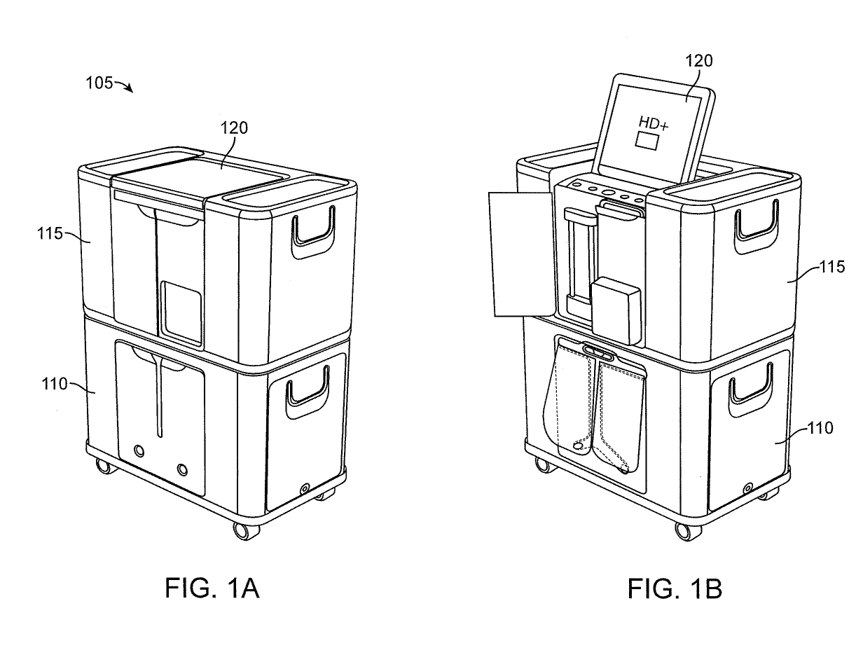

FIG. 1A shows a dialysis system 105 that is configured to perform dialysis on a patient. The dialysis system 105 includes two or more individual modules that can be coupled to one another in an arrangement that collectively forms the dialysis system, as described in detail below. The dialysis system 105 can include a variety of different modules that can be coupled in various configurations that are adapted to different environments. For example, an in-center configuration of modules is particularly adapted for use in a dialysis center, while a home configuration is adapted for use in a user's home. Other configurations are possible.

In the embodiment of FIGS. 1A and 1B, the modules include a first module 110 and a second module 115 that can be removably coupled to the first module 110 to collectively enable the dialysis system 105 to perform dialysis. In an embodiment, the first module 110 includes one or more subsystems that enable the first module 110 to perform water treatment and dialysate preparation, as described more fully below. The second module 115 includes one or more subsystems that enable the first module to perform an extracorporeal procedure on the patient, such as dialyzing a patient's blood using dialysate from the first module 110. The dialysis system 105 also includes a third module comprised of a user interface module 120 that enables a user to interact with the dialysis system 105. FIG. 1A shows the dialysis system 105 in an off state wherein the user interface module 120 is configured in a stand-by mode. FIG. 1B shows the dialysis system 105 in an active state wherein the user interface module 120 is configured for use.

It should be appreciated that the dialysis system shown in FIGS. 1A and 1B is exemplary and that variations form the configuration shown in FIG. 1A and 1B are within the scope of this disclosure. The dialysis system 105 can include a variety of modular configurations that vary from what is shown in FIGS. 1A and 1B. For example, the first module 110 can include various other subsystems and can be configured to perform procedures other than water treatment and dialysate preparation. Alternatively, the first module could be divided into two or more sub-modules, with individual modules designed for various functions, e.g., water treatment, dialysate mixing, etc. Likewise, the second module 115 can be configured to perform procedures other than dialysis. The arrangement and interchangeability of the modules may vary from the examples described herein.

Some exemplary configurations of subsystems of a dialysis system are described herein for purpose of example, although it should be appreciated that the configuration of the dialysis system 105 can vary. In an embodiment, the dialysis system 105 includes a plurality of subsystems that collectively operate to (1) receive and purify water; (2) use the water to prepare dialysate; and (3) supply the dialysate to a dialyzer module that may perform various types of dialysis on the blood of a patient such as hemodialysis, ultrafiltration and hemodiafiltration. The dialysis system includes plumbing that provides fluid pathways for water, dialysis, and blood to flow through the dialysis system, as well as one or more pumps that interface with the plumbing for driving fluid flow through the system. The dialysis system can also include one or more sensors, such as fluid flow sensors, pressure sensors, conductivity sensors, etc. for sensing and reporting one or more characteristics of fluid flowing through the system.

In an embodiment, the entire dialysis system (including the water preparation and purification system, dialysate preparation system, flow balancer system, dialyzer, and hardware, such as plumbing and sensors) is formed of at least one housing that is compact and portable. The housing may be collectively formed of a plurality of modular housings that are coupled to one another. In addition, the dialysis system can prepare dialysate using a tap water, such as tap water from a home or hotel room. In an embodiment, the entire dialysis system, including all of the elements described above, consumes less than about 22″ by 14″ by 9″ of space when dry, which generally corresponds to the size limit for carry-on baggage of an airline. In an embodiment, the entire dialysis system weighs less than about fifty pounds when dry.

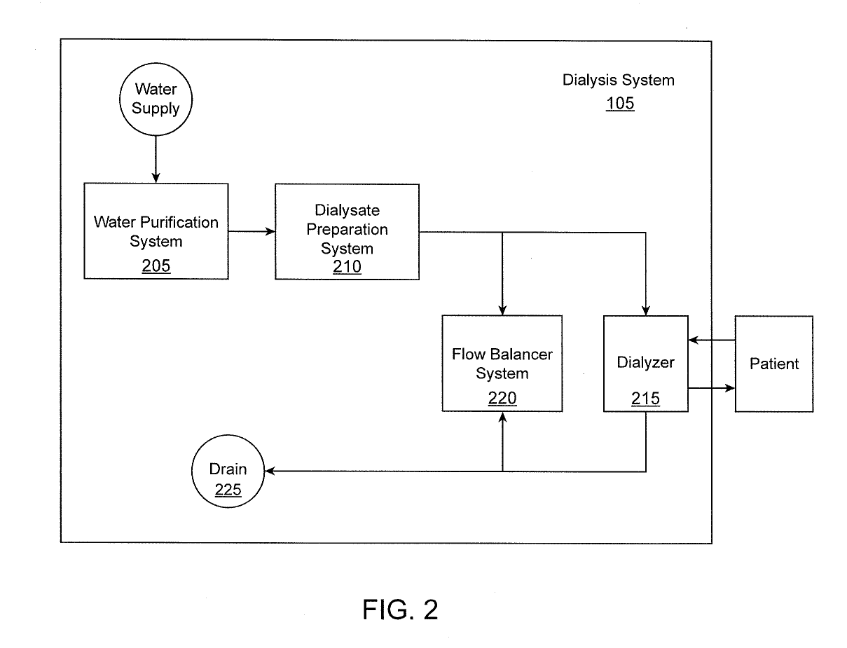

FIG. 2 shows a high level, schematic view of an exemplary dialysis system that can be controlled using a user interface system. The dialysis system includes a water preparation and purification system 205 that purifies water from a water supply 7. The water purification system 205 supplies the purified water to a dialysate preparation system 210 that uses the purified water to prepare dialysate. The dialysis system further includes a dialyzer 215 that receives the dialysate from the dialysate preparation system 210 and performs one or more of the various forms of dialysis on a patient's blood. In an embodiment, the dialyzer 215 and the dialysate preparation system 210 both interface with a flow balancer system 220 that regulates the flow of dialysate to the dialyzer to achieve different types of dialysis, including hemodialysis, ultrafiltration, and hemodiafiltration.

Diffusion is the principal mechanism in which hemodialysis removes waste products such as urea, creatinine, phosphate and uric acid, among others, from the blood. A differential between the chemical composition of the dialysate and the chemical composition of the blood within the dialyzer causes the waste products to diffuse through a membrane from the blood into the dialysate. Ultrafiltration is a process in dialysis where fluid is caused to move across the membrane from the blood into the dialysate, typically for the purpose of removing excess fluid from the patient's blood stream. Along with water, some solutes are also drawn across the membrane via convection rather than diffusion. Ultrafiltration is a result of a pressure differential between a blood compartment and a dialysate compartment in the dialyzer where fluid moves from a higher pressure to a lower pressure across a semi-permeable membrane. In some circumstances, by design or unintentional consequence, fluid in the dialysate compartment is higher than the blood compartment causing fluid to move from the dialysate compartment into the blood compartment. This is commonly referred to as reverse ultrafiltration.

In hemodiafiltration, a high level of ultrafiltration is created, greater than the amount required to remove fluid from the patient's blood, for the purpose of increasing convective solute transport across the membrane. The amount of fluid in excess of what is required to be removed from the patient's blood must therefore be returned to the blood stream in order to avoid an adverse hemodynamic reaction. This is accomplished by intentionally increasing the pressure in the dialysate compartment of the dialyzer to cause the appropriate amount of reverse ultrafiltration. This process of ultrafiltration alternating with reverse ultrafiltration is often referred to as “push-pull hemodiafiltration.” This is a significant improvement over more common methods of hemodiafiltration where sterile fluid is administered to the patient in a location outside of the dialyzer.

In use, the patient is coupled to the dialyzer 215 such that the patient's blood flows into and out of the dialyzer 215 using devices and techniques known to those skilled in the art. The patient or clinician can interact with the user interface module 120 to control one or more aspects of the dialysis system and to also receive feedback from the dialysis system 105 during use. The dialysis system prepares dialysate using water from a household water source, such as a tap, that has been previously prepared through filtration and purification before being mixed with various dialysate components to make the dialysate, and then flows the dialysate through the dialyzer in communication with the blood such that one or more of the dialysis processes on the blood is performed. The water purification system includes a plurality of subsystems that collectively operate to purify the water including pasteurization of the water. The purified water is then mixed with dialysate concentrates to form dialysate, which is supplied to the dialyzer 215 and to the flow balancer system, which regulates the flow of dialysate to the dialyzer 215 to selectively achieve different types of dialysis, including hemodialysis, ultrafiltration, and hemodiafiltration, as described more fully below. The dialysis system supplies the used dialysate to a drain 225. In an embodiment, the system recaptures heat from the used dialysate before the used dialysate is sent to the drain.

The following pending U.S. Patent Applications (which are incorporated herein by reference in their entirety) describe exemplary embodiments of dialysis systems and subsystems: (1) U.S. patent application Ser. No. 12/795,444 entitled “Dialysis System”; (2) U.S. patent application Ser. No. 12/795,498 entitled “Dialysis System with Ultrafiltration Control”; (3) U.S. patent application Ser. No. 12/795,371 entitled Microfluidic Devices; and (4) U.S. patent application Ser. No. 12/795,382 entitled “Fluid Purification System”.

### Dialysis System: Modular Configuration

As discussed, the dialysis system 105 is configured as two or more modules that can be coupled to one another in one or more arrangements that collectively form the dialysis system. Each module comprises one or more subsystems of the dialysis system 105, such as one or more of the exemplary subsystems shown in FIG. 2.

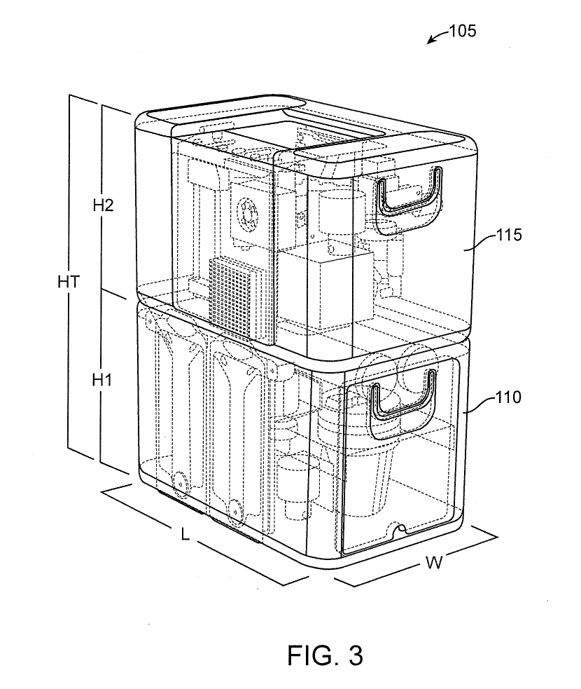

FIG. 3 shows exemplary embodiments of a first module 110 coupled to a second module 115 with the outer housings of the modules illustrated as transparent in order to show exemplary components contained within the housings. The user interface module 120 is not shown in FIG. 3. In the illustrated embodiment, the first module 110 is a water treatment and dialysate preparation module that includes components configured to treat water (such as using an ultrapasteurization process) and to use the treated water to prepare dialysate. As mentioned, the first module 110 is configured to treat a domestic water source, such as tap water from a home or hotel. The second module 115 is an extracorporeal module that includes components configured to perform dialysis on a patient's blood. The first and second modules are described in detail below.

At least some of the modules, such as the first and second modules, are formed of an outer housing that defines an internal cavity sized and shaped to house one or more hardware components of a particular dialysis subsystem. In an exemplary embodiment as shown in FIG. 3, each housing is a rectangular-shaped housing with the housing of the first module 110 configured to support the housing of the second module 115. That is, the second module 115 can be positioned atop the first module 110 in a stacked configuration such that the first and second modules mechanically, fluidly and/or communicatively couple to one another. In an embodiment, the system automatically commences an disinfection or sanitation process upon coupling of the modules in order to eliminate any unsanitary elements in the system upon coupling of the modules. The outer housing may be made of any of a variety of materials. In an embodiment, the outer housing is made of a hard material, such a hard plastic or metal, configured to withstand loads and protect the inner components. In another embodiment, the outer housing is made of a softer material that may be lighter to facilitate lifting of the module. The shape of the outer housing of each module can vary.

Each module has a weight such that the module is configured to be lifted by an average user when the module is dry. As shown in FIG. 3, each module has a length L, a width W, and a height H (H1 for the first module 110 H2 for the second module 115). When stacked, the first and second modules collectively provide the dialysis system with a total height HT. FIG. 3 shows the first and second modules having the same or substantially same lengths and widths although the dimensions can vary between modules. In an exemplary embodiment, each module has a length of about 19.5 inches, a width of about 12 inches, and a height of about 12 inches such that when stacked the modules collectively have a height of about 24 inches, although the dimensions may vary. In an embodiment, each of the modules weighs less than twenty-five pounds when dry.

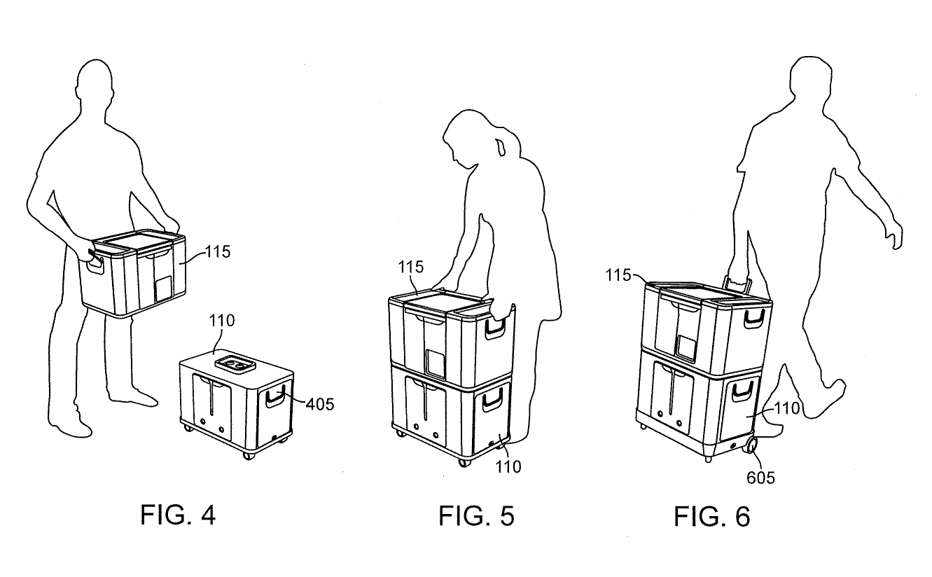

As shown in FIGS. 4 and 5, each of the first and second modules 110 and 115 is sized and shaped such that each can be carried and transported by an average male or female user. In this regard, each of the first and second modules 110 and 115 may include items such as one or more handles that facilitate the user grabbing onto and holding the modules. FIG. 4 shows the first module 110 and second module 115 coupled to one another in a stacked relationship. In an embodiment, the first module 110 is a stationary in-center module such that it is configured to be positioned in a stationary, fixed position and is not transportable. In such a configuration, the first module 110 may be fixedly attached to a supply of water. In another embodiment, the first module 110 is transportable such that a user can carry, roll, or otherwise transport the first module 110 between two or more locations. The dialysis system 105 may also include a plurality of first modules 110, including a stationary first module 110 that is located at a dialysis center and a transportable first module 110 that is configured to be transported. This permits the user to interchange the second module or any other modules with any of a variety of first modules. In an embodiment, the modules may be configured to permit in-center, daily-short and/or nocturnal treatments using a common platform module and exchanging one or more modules, such as a water treatment module, that permits treatment in a unique environment. For example, a water treatment module may be configured to allow short-to-long duration treatments.

FIG. 5 shows the first module 110 and second module 115 uncoupled from one another with a user holding the second module 115. The bottom-most module may be configured to facilitate transport of the modules when coupled to one another. For example, as shown in FIG. 6, the bottom-most module may include one or more pairs of wheels 605 that permit the user to roll the dialysis system from one location to another location. Or, the bottom-most module may be configured to be positioned in a cart having wheels.

Some examples of modules are now described. As mentioned, the type, quantity and interchangeability of the modules of the dialysis system can vary from the examples described herein.

User Interface Module

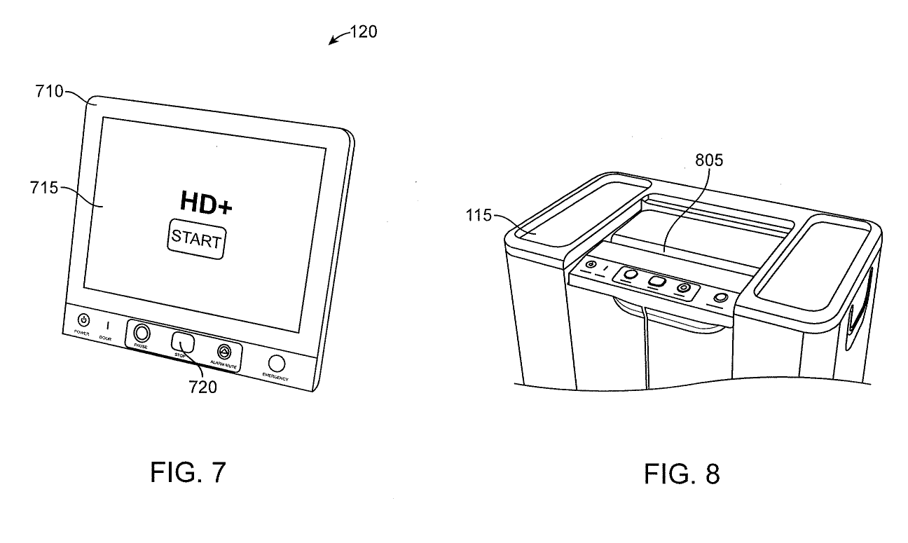

The user interface module 120 is configured to enable a user to interact with the dialysis system 105, such as to input commands to the system and to receive feedback from the system. FIG. 7 shows an exemplary embodiment of the user interface module 120 comprised of a tablet 710 that is sized and shaped to be held by a user. The tablet 710 can vary in size. For example, the tablet can be sized and shaped to be held by a user. In another embodiment, the tablet is sized and shaped to be stored in a pocket of a user's clothing. The user interface module 120 is described herein in the context of being a tablet although configurations of other than tablets are within the scope of this disclosure.

The tablet 710 has a display 715 that is configured to display any of a variety of alphanumeric or graphic images to the user. The display 315 may incorporate a touch screen. The tablet 710 also includes one or more user input elements 720, such as hard keys and/or soft keys. The user input elements 720 may include hard keys or buttons, such as an alphanumeric keypad or other buttons dedicated to specific tasks. The user input elements 720 may also include virtual buttons that are accessed via a touch screen. The user interface module 120 includes a controller (which may be housed within the tablet 710) that is adapted to communicate with and control one or more of the subsystems (FIG. 2) of the dialysis system 105. The controller can be any type of computer controller or central processing unit (CPU) adapted to receive and process instructions and submit commands. The controller interfaces with computer-readable software code that resides in internal memory. The code can be loaded onto the computer and modified via an input-output interface of the system. U.S. patent application Ser. No. 61/418,753, entitled “DIALYSIS SYSTEM USER INTERFACE”, describes an exemplary user interface for a dialysis system and is incorporated herein by reference in its entirety.

The user interface module 120 also includes one or more indicators configured to provide a visual and/or audio signal to the user. The visual or audio signal may relate to any aspect of the dialysis system, such as the operational state of the dialysis system 105 or an alarm or error situation. The indicators may include speakers and lights. The indicators may also include haptics that provide tactile feedback to the user. The indicators may also include a wireless transmitter that is configured to provide a wireless signal to a user or clinician, such as via a text message, telephone call, email, etc.

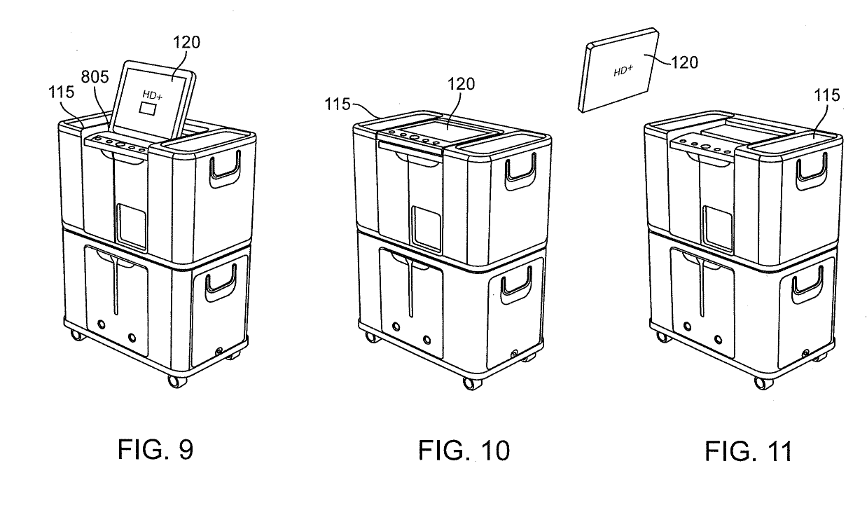

The user interface module is communicatively coupled to one or more of the other components of the dialysis system 105 via a hardwired or wireless communication pathway. For example, as shown in FIG. 8, the second module 115 may include a port 805 that is sized and shaped to receive at least a portion of the tablet 710 such that the tablet 710 can be plugged or seated in the port 805. When the tablet 710 is seated in the port 805, as shown in FIG. 9, the user interface module 120 communicatively couples to the second module 115 and/or the first module 110 via a hardwired connection, such as via an RS-232 connection or other appropriate connection. When properly positioned in the port 805, the tablet 710 is disposed in an upright orientation. As shown in FIG. 10, the tablet 710 may also be positioned in a flat orientation on the second module 115. As mentioned, a wireless connection can also be used to provide communication between the user interface module 120 and the other modules, as shown in FIG. 11. Any wireless protocol may be used to provide communication, including Bluetooth, WiFi, etc.

Water Treatment Module

In an embodiment, the first module 110 is configured to treat water and use the treated water to prepare dialysate, although each of these functions may be divided into separate modules that are operatively coupled together during use. In this regard, the first module 110 includes components of the water purification system 205 and the dialysate preparation system 210 (FIG. 2). For example, the water purification system may include components such as a microfluidic heat exchange (HEX) system adapted to achieve pasteurization of the liquid passing through the fluid purification system. The fluid purification system may also include one or more additional purification subsystems, such as a sediment filter system, a carbon filter system, a reverse osmosis system 125, an ultrafilter system, an auxiliary heater system, a degassifier system, or any combination thereof. The fluid purification system may also include hardware and/or software to achieve and control fluid flow through the fluid purification system. The hardware may include one or more pumps or other devices for driving fluid through the system, as well as sensors for sensing characteristics of the fluid and fluid flow.

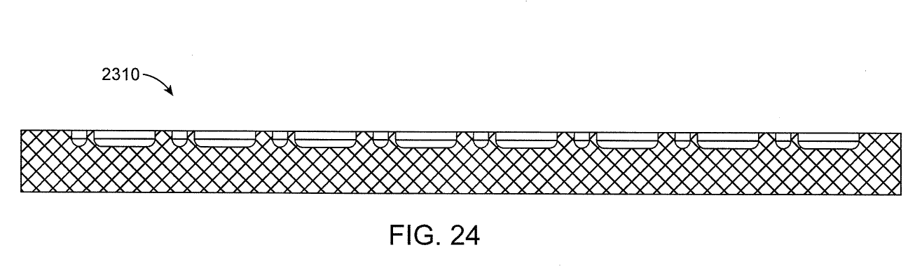

The dialysate preparation system 210 may include components such as an acid pump 170 that fluidly communicates with a supply of concentrated acidified dialysate concentrate for mixing with the purified water. The water flows from the water purification system to the acid pump, which pumps the acid concentrate into the water. The water (mixed with acid) then flows into a first mixing chamber, which is configured to mix the water with the acid such as by causing turbulent flow. FIG. 23 shows a top-down view of an exemplary flow pathway that forms a mixing chamber assembly 2310 for mixing ultra-pure water with an acid concentrate. The flow pathway may be formed on a layer of material with a plurality of layers arranged in stack. FIG. 24 shows a cross-sectional view of an exemplary layer. The mixing chamber assembly 2310 forms an undulating pathway and a plurality of mixing chambers positioned intermittently along the pathways through which the water and the acid concentrate flow. The undulating pathway undergoes a series of variations in diameter and/or depth (or other dimension) that achieves more mixing conditions in the flow than would otherwise be achieved with uniform dimension along the length of the flow pathway. The mixing conditions are localized variations in flow velocity and flow direction particularly in the regions where the diameters of the flow pathways undergo change. This causes localized mixing of the water and acid concentrate. In an embodiment, the system includes two mixing chambers arranged in series wherein a first mixing chamber mixes acid concentrate and water to produce a diluted acid. The diluted acid then flows into a second mixing chamber where it is mixed with a sodium bicarbonate. In an embodiment, the mixing chambers are about 3 millimeters wide and the pathways that connect the mixing chambers are about 1.5 millimeters wide, although the dimensions may vary.

From the mixing chamber, the acid-water mixture flows toward a bicarbonate pump. A sensor, such as a conductivity sensor, may be positioned downstream of the first mixing chamber for detecting a level of electrolytes in the mixture. The conductivity sensor may be in a closed loop communication with the acid pump and a control system that may regulate the speed of the acid pump to achieve a desired level of acid pumping into the water. The aforementioned components are examples and it should be appreciated that the configuration of the dialysate preparation system can vary.

Exemplary embodiments of a water purification system and a dialysate preparation system are described in the following co-pending U.S. patent application Ser. Nos. which are incorporated herein by reference in their entirety: (1) U.S. patent application Ser. No. 12/795,444 entitled “Dialysis System”; (2) U.S. patent application Ser. No. 12/795,498 entitled “Dialysis System with Ultrafiltration Control”; (3) U.S. patent application Ser. No. 12/795,371 entitled Microfluidic Devices; and (4) U.S. patent application Ser. No. 12/795,382 entitled “Fluid Purification System”.

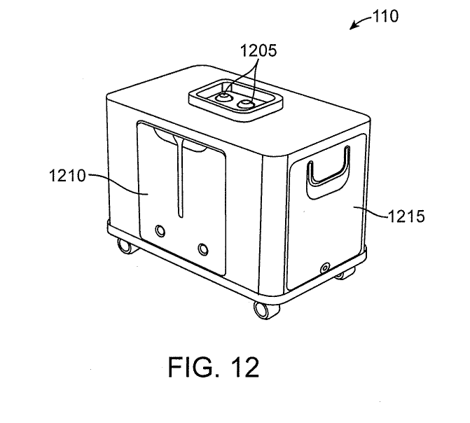

As shown in the semi-transparent view of FIG. 3, all of the hardware components of the water purification system 205 and the dialysate preparation system 210 are contained within the outer housing of the first module 110. With reference now to FIG. 12, there is shown a perspective view of an exemplary embodiment of the first module 110, which includes one or more coupling elements 1205 that are configured to couple to corresponding coupling elements on the second module 115 to permit mechanical, fluid and/or electrical communication therebetween. In the illustrated embodiment, the coupling elements 1205 are fluid plumbing connections that connect to corresponding fluid plumbing connections on the second module 115. The fluid plumbing connections are fluidly connected to internal plumbing of the modules to permit passage of fluids, such as water or dialysate, from one module to another module. In an embodiment, the plumbing connection of the second module 115 (the top module) couples to the plumbing connection of the first module (the bottom module) when the second module 115 is positioned atop the first module 110. The weight of the second module 115 secures the plumbing connections together. A clamping mechanism between the plumbing connections can be configured to lock the modules together, so that once the modules are connected, the modules cannot accidentally become dislodged or disconnected.

In an embodiment, the system includes one or more sensors that communicate with the user interface module such as in a feedback arrangement. The user interface module is configured to initiate one or more alarms, indicator lights, control overrides, etc. that indicate (i) whether the modules have been connected together correctly, and/or (ii) whether the connection between modules somehow becomes suspect or damaged during a procedure.

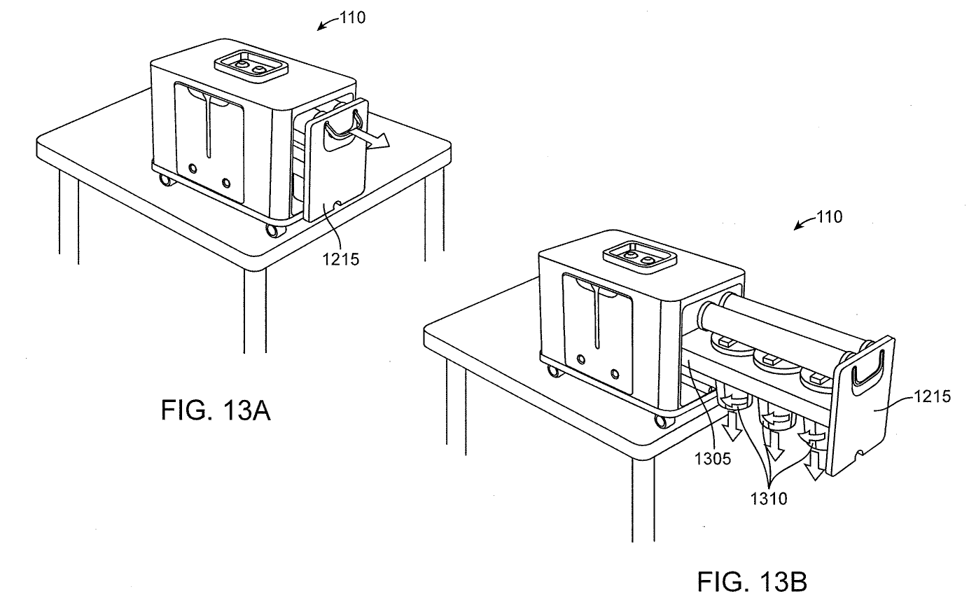

With reference still to FIG. 12, any of the modules may include doors 1210, drawers 1215, or other hardware component on the outer housing that may be opened to provide access to the internal components of the modules. For example, as shown in FIGS. 13A and 13B, the first module 110 has a drawer 1215 that a user may slide outward from the outer housing to expose internal components of the first module 110. In the illustrated embodiment, the drawer 1215 slides outward to expose a rack 1305 on which hardware components, such as one or more water filters 1310, are removably mounted. When the user opens the door 1215 and slides out the rack 11305, the water filters 1310 are automatically disengaged from other internal components of the first module 110. A user can inspect, maintain, replace, etc. the internal components of the first module (or any module) by simply opening a corresponding door or drawer on the module.

The first module 110 may also include coupling components that permit connection to an external source of water and/or to an external drain. For example, one or more valves or water pipe couplings may be positioned on or within the external housing of the first module 110 to permit fluid hoses to be fluidly coupled to the internal plumbing of the first module 110.

Extracorporeal Module

The second module 115 is configured to perform an extracorporeal procedure, such as dialysis, on the patient. In this regard, the second module 115 includes components that enable the procedure. Where the extracorporeal procedure is dialysis, the components include the flow balancer system 220 (FIG. 2) and the dialyzer 215 (FIG. 2), although the dialyzer may occupy its own module and be coupleable to a module comprising the flow balance system. Exemplary embodiments of the flow balancer system 220 and the dialyzer 215 are described in co-pending U.S. patent application Ser. No. 12/795,444 entitled “Dialysis System”, U.S. patent application Ser. No. 12/795,498 entitled “Dialysis System with Ultrafiltration Control”, and U.S. patent application Ser. No. 12/795,371 entitled “Microfluidic Devices”, which are incorporated herein by reference in their entirety. As shown in the semi-transparent view of FIG. 3, all of the hardware components of the flow balancer system 220 and the dialyzer 215 are contained within the outer housing of the second module 115. Such components may include plumbing for fluid flow (and corresponding inlets and outlets), valves, pumps, and components of the dialyzer.

The second module 115 also includes one or more coupling elements that permit coupling of the second module 115 to the other modules. For example, as described above with reference to FIG. 8, the second module 115 includes a port 805 that is configured to receive at least a portion of the tablet 710 for coupling the tablet 710 to the second module 115. The second module 115 may also include one or more doors, drawers, or other components that provide or enhance accessibility to components of the second module 115.

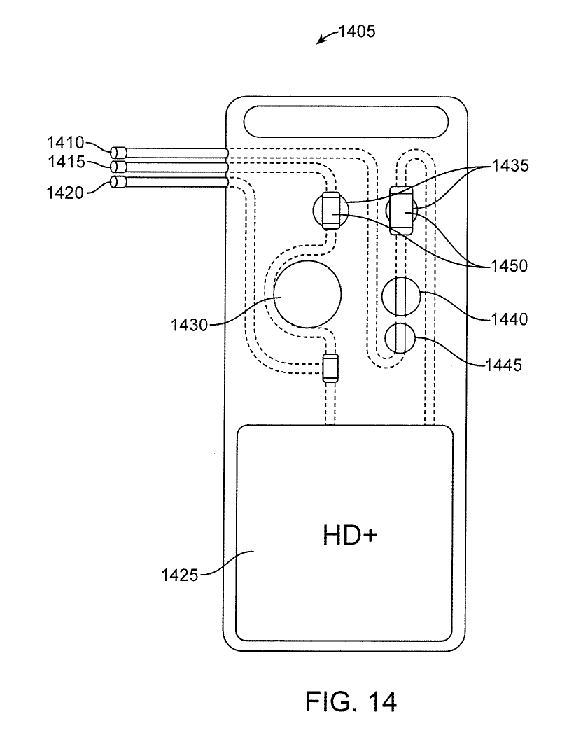

In an embodiment, the second module 115 is configured to be removably interfaced with components that are used pursuant to the extracorporeal processing such as dialysis. For example, as shown in FIGS. 14-17, the second module 115 may removably interface with a cartridge module 1405. FIG. 14 shows a plan view of an exemplary cartridge module 1405. The cartridge module 1405 includes plumbing, such as pipes, cables, tubing, etc., through which fluid flow can occur. The plumbing includes outlets that can be fluidly coupled to corresponding inlets or outlets in the second module. A user can couple the cartridge module 1405 to the second module 115, such as in a “plug-in” manner to enable the cartridge to interface with the components of the second module 115.

In the embodiment of FIG. 14, the plumbing of the cartridge module 1430 includes a venous line 1410 that is for the flow of blood to the patient's dialysis access system such as a fistula, graft or catheter. The cartridge module 1405 also includes an arterial line 1415 for the flow of blood from the patient's dialysis access system, and a heparin line 1420 that combines with the arterial line 1415. The flow lines communicate with a dialyzer 1425. In addition, the cartridge module 1405 includes one or more ports that are configured to receive additional components that facilitate processing of the patient's blood. Such ports can include, for example, a pump port 1430 that receives a fluid pump, a pressure sensor port 1435 that receives a pressure sensor, and other ports such as a line clamp port 1440 or air detection port 1445. Any of a variety of additional hardware components may be incorporated into the cartridge module 1405, such as one or more drip bulbs 1450.

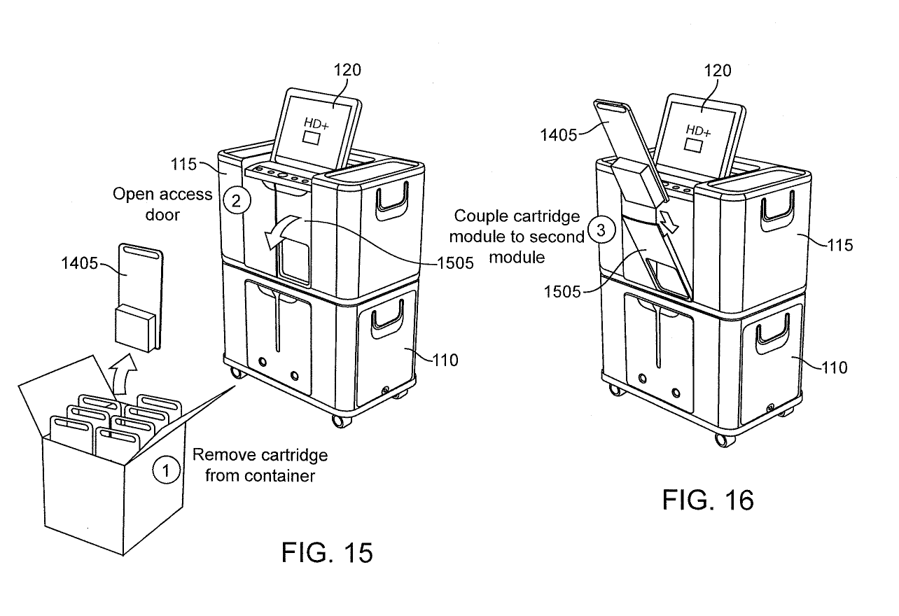

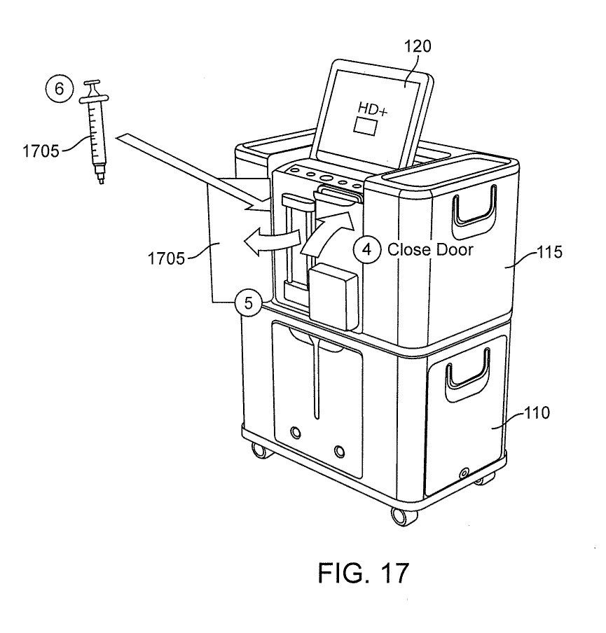

FIGS. 15-17 show the steps of an exemplary method of removably coupling the cartridge module 1405 to the second module 115 of the dialysis system. As shown in FIG. 15, the cartridge module 1405 may initially be stored in a collection of cartridge modules stored within a container 1505. In an initial Step 1 in FIG. 15, the user removes a cartridge module 1405 from the container 1505. Still with reference to FIG. 15, in a subsequent Step 2, the user opens a cartridge access door 1505 on the second module 115 to provide access to a port or seat in which the cartridge module 1405 can be seated for coupling the cartridge module 1405 to the system. With reference to FIG. 16, in Step 3, the user then couples the cartridge module 1405 to the second module 115, such as by slidingly engaging the cartridge module 1405 into a port in or on the access door 1505. Once the cartridge module 1405 is coupled to the second module 115, the user can close the door 1505, as shown in Step 4 of FIG. 17. The module 115 may also be configured to receive any additional items that are necessary for dialysis or that facilitate dialysis, such as a dosage of heparin. As shown in Step 5 of FIG. 17, the second module 115 may include a second access door 1705 that can be opened to provide a dosage of heparin to the second module 115. The user opens the second access door 1705 and inserts the heparin dosage into the second module 115.

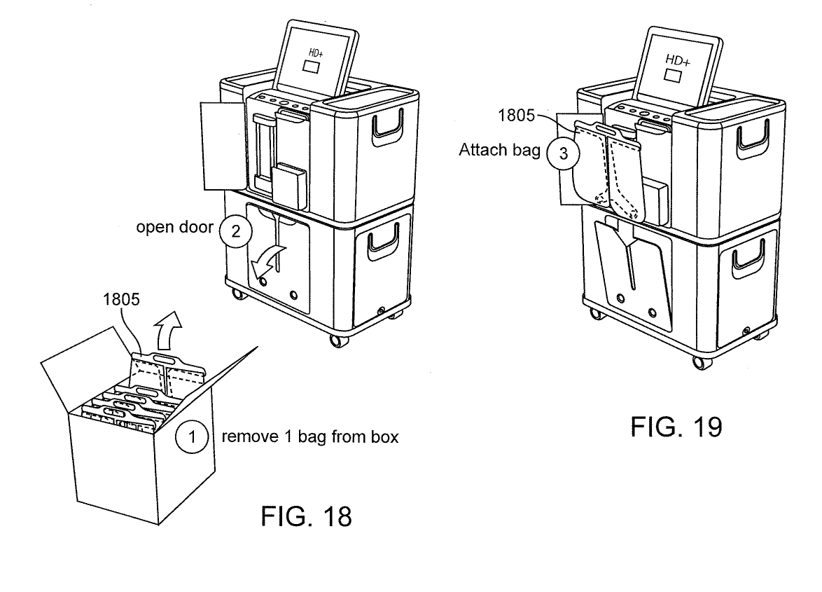

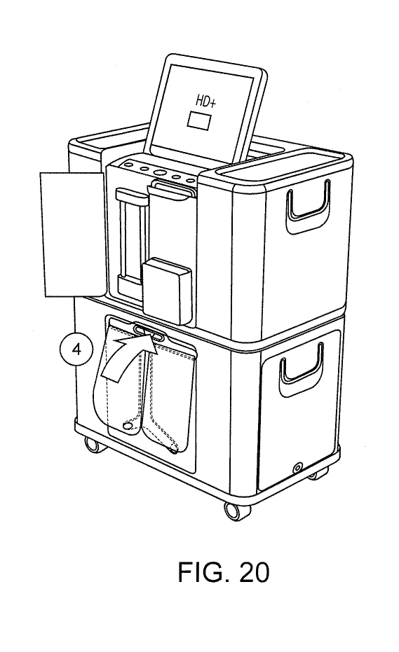

With reference now to FIGS. 18-20, the first module 110 may also be configured to removably interface with one or more additional modules, such as bag modules that contain supply of material to assist in dialysis. As shown in FIG. 18, a bag module 1805 that contains materials (such as chemicals) used for preparing dialysate may be stored in a bag module container. In Step 1 of FIG. 18, the user removes a bag module 1805 from the container. The door 1215 of the first module 115 is configured to be removably coupled to the bag module 1805. The user opens the door 1215 (as shown in Step 2 FIG. 18) and then attaches the bag module 1805 to the door 1215, as shown in Step 3 of FIG. 19. Once attached, the door 125 is closed with the bag module 1805 positioned in place, as shown in Step 4 of FIG. 20.

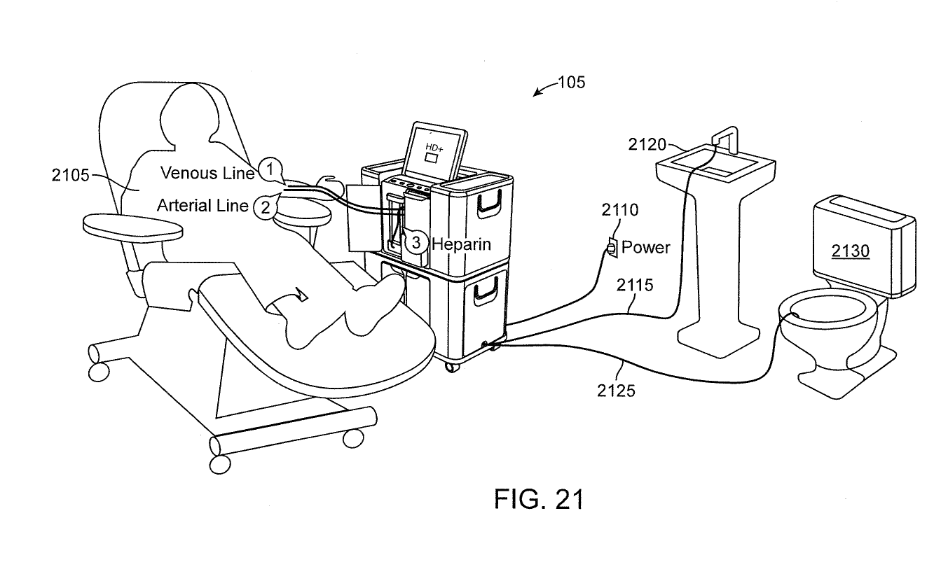

Once the first and second modules are coupled together, the user can couple the user interface module to the dialysis system for operation. FIG. 21 shows the dialysis system 105 with the first and second modules coupled to one another and the user interface module coupled to the system. The user 2105 is attached to a venous line and an arterial line for performing dialysis on the user. A source of power, such as a home power outlet 2110, is attached to the dialysis system 105. In addition, a source water conduit 2115 is coupled to the dialysis system 105 and to a source of water, such as a home water tap 2120, for supplying water to the dialysis system 105. A waste conduit 2125 is coupled to the dialysis system 105 and to a drain, such as a home toilet 2130. With the system configured as shown in FIG. 21, the user can use the dialysis system 105 for performing a dialysis procedure at home. As mentioned, other configurations are possible, such as a configuration particularly suited for use in a dialysis center.

### Additional Embodiment of Dialysis System

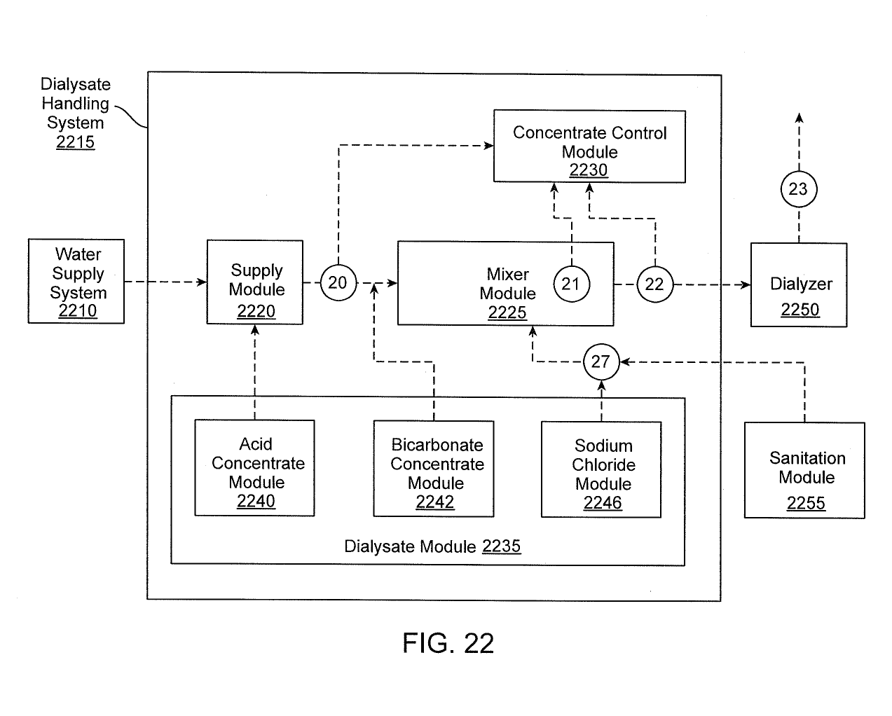

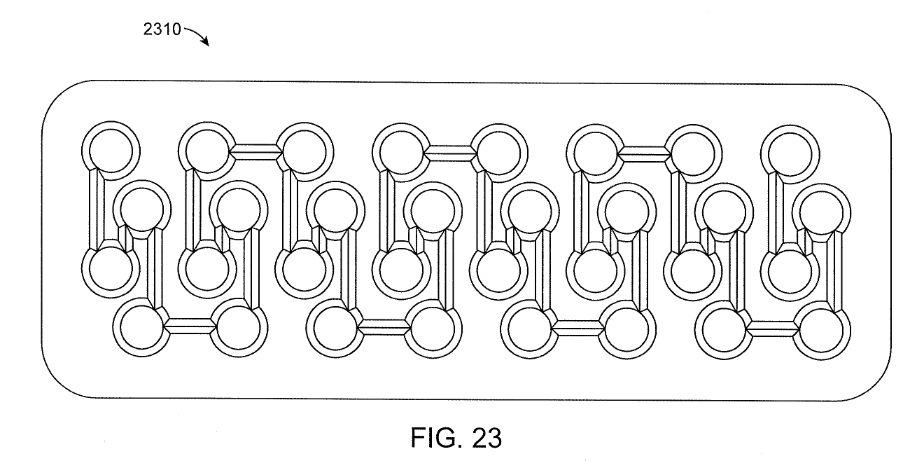

FIG. 22 shows an additional embodiment of a modular dialysis system. Any of the features of the embodiment of FIG. 22 may be incorporated or otherwise combined with the previously described embodiments. The dialysis system shown in FIG. 22 comprises a water supply system 2210 and a dialysate handling system 2215. The water supply system 2210 supplies filtered purified water to the dialysate handling system 2215. In an embodiment, the dialysate handling system 2215 comprises a supply module 2220, a mixer module 2225, a concentrate control module 2230, and a dialysate module 2235. The dialysate module 2235 comprises one or more modules, such as an acid concentrate module 2240, a bicarbonate concentrate module 2242, and in one embodiment, a sodium chloride concentrate module 2246. In another embodiment, the sodium chloride module is combined into the acid concentrate module 2240. The dialysate handling system 2215 prepares the dialysate to a predetermined chemistry and supplies the dialysate to the dialyzer 2250.

The various modules are interconnected by plumbing to permit fluid to flow between the various modules. The supply module 2220 is provided with acid from the acid concentrate module 2240 of the dialysate module 2235 such that the supply module 2220 outputs a dilute acid solution. The dilute acid solution combines with a bicarbonate solution provided by the bicarbonate concentrate module 2242 of the dialysate module 2235, and the resulting acid/bicarbonate solution is provided to the mixer module 2225. The sodium chloride concentrate module 2246 of the dialysate module 2235 provides a sodium chloride (NaCl) solution to the mixer module 2225. The acid/bicarbonate solution is further mixed with the sodium chloride solution in the mixer module 2225 which outputs the resulting dialysate of a predetermined chemistry to the dialyzer 2250. The dialyzer 2250 dialyzer is configured to perform various types of dialysis on the blood of a patient such as hemodialysis, ultrafiltration and hemodiafiltration.

A solute concentration sensor 20 is provided in the plumbing flow stream after the supply module 2220. The pH sensor 20 is configured to sense solute concentration data and communicate the pH data to the concentrate control module 2230. The concentrate control module 2230 is configured to provide control data to one or more of the other modules. In this regard, the concentrate module 2230 can be configured in a feedback relationship with any of the other modules. In an embodiment, the concentrate control module 2230 controls the acid concentrate module 2240 so as to adjust the solute concentration of the solution exiting the supply module 2220 to a predetermined value.

An additional solute concentrate sensor 21 is provided within the mixer module 2225. The alkalinity sensor 21 senses and communicates an solute concentration value to the concentrate control module 2230. The concentrate control module 2230 is configured to control the bicarbonate concentrate module 2242 so as to adjust the solute concentration of the solution exiting the mixer module 2225 to a predetermined value. An additional solute concentration sensor 22 is provided after the mixer module 2225. The sodium chloride sensor 22 senses and communicates solute concentration to a concentrate monitoring module. The concentrate monitoring module monitors the total solute concentration, and alerts the system when the solute concentration is unsafe, opening a bypass valve and directing the unsafe dialysate to the drain. Solute concentration sensors 20, 21 and 22 are known in the art, and include, but are not limited to, electric conductivity sensors or photometric sensors. It should be appreciated that any of a variety of other sensors can be positioned within the system.

The dialysis system of FIG. 22 further comprises a sanitation module 2250. The sanitation module 2250 provides a sanitizing solution comprising a sanitizing agent, such as, but not limited to, sodium hypochlorite, and a tracer agent, such as, but not limited to, sodium chloride (NaCl), to the mixer module 2225. The sanitizing agent provides for sanitizing the flow stream of the dialysis system, such as, but not limited to, the dialysis flow plumbing or passages through the dialyzer 2250. The tracer agent provides for detecting whether the sanitizing solution is present and sufficiently rinsed out of the dialysis system. A relative concentration of the tracer agent between the sanitation solution and the purified water rinse is used to determine whether a sufficient concentration of sanitation solution is present in the flow stream during the sanitation process and whether the flow stream is sufficiently flushed of sanitation solution during the rinse process.

The solute concentration sensor 22 is utilized to detect the presence of sodium chloride during a sanitizing and rinse process by way of detecting electrical conductivity of the solution present in the flow stream. A valve 27 is disposed in the plumbing between the sanitation module 2255 and the mixer module 2225. The valve 27 is adapted to control the supply of solution provided to the mixer module 2225 by the sodium chloride concentrate module 2246 and the sanitation module 2250. During a sanitation process, the valve 27 may be used to stop the flow from the sodium chloride concentrate module 2246 and allow the flow from the sanitation module 2250. During the rinse process, the valve 27 may be used to stop the flow from the sanitation module 2250 and the sodium chloride concentrate module 2246. During startup and dialysis, the valve 27 may used to allow the flow from the sodium chloride concentrate module 2246 and not allow the flow from the sanitation module 2250.

In another embodiment, the sodium chloride concentrate module 2246 is configured to be removed from the dialysate module 2235 and replaced by the sanitation module 2250 in anticipation of a sanitation process. The sanitation module 2250 is configured to be removed from the dialysate module 2235 and replaced by the sodium chloride concentrate module 2246 in anticipation of a rinse and dialysis process. The physical swap-out of the sodium chloride concentrate module 2246 and the sanitation module 2250 provides a level of safety that reduces the likelihood of the sanitation module 2250 providing sanitation solution to the dialyzer 2250 during the rinse and dialysis processes as the sanitation module 2250 is not coupled to the dialysis system during those processes.

In an embodiment, a sodium chloride sensor 23 is provided downstream of the dialyzer 2250. The sodium chloride sensor 23 is utilized to detect the presence of sodium chloride during the sanitizing and rinse process in substantially the same way as solute concentration sensor 22. In an embodiment, the dialysis system comprises only solute concentration sensor 20. In another embodiment, the dialysis system comprises only solute concentration sensor 22. In another embodiment, the dialysis system comprises both solute concentration sensor 20 and 22.

In accordance with an embodiment of a method for sanitizing components of a dialysis system, a predetermined sanitizing solution comprising a sanitizing agent, such as, but not limited to, sodium hypochlorite, and a tracer agent, such as, but not limited to, sodium chloride (NaCl) is supplied to a respective flow stream for a predetermined amount of time suitable for sanitizing the flow stream. Subsequently, purified water is supplied to the respective flow stream for a predetermined amount of time suitable for flushing out the sanitizing solution from the respective flow stream. An electrical conductivity sensor suitable for detecting the presence of the tracer agent is positioned in the flow stream. The duration of the purified water flush is predetermined wherein the electrical conductivity sensor detects a predetermined concentration of the tracer agent in the flow stream signifying that sanitizing solution is sufficiently rinsed from the respective flow stream.

It is understood that tracer agents other than sodium chloride may be utilized to change the electrical conductivity of the sanitizing solution so as to permit the measure of relative difference in conductivity between the sanitizing solution and the purified water, suitable for determining whether the sanitizing solution is sufficiently flushed or rinsed from the respective flow stream. It is understood that sanitizing agents other than sodium hypochlorite may be utilized to sanitize the flow stream.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A modular dialysis system, comprising:
a plurality of modules adapted to be operatively removably coupled together to collectively form a dialysis system capable of performing a dialysis procedure on a patient, the modules including:
a user interface module comprising at least one user input element and at least one display element;
a water treatment module comprising water treatment components configured to treat water for use in the dialysis procedure; and
a dialysis module comprising components configured to perform dialysis.

2. The system of claim 1, wherein the water treatment module is configured to prepare dialysate.

3. The system of claim 2, wherein the water treatment module is adapted to deliver dialysate to the dialysis module when the water treatment module is attached to the dialysis module.

4. The system of claim 1, wherein the dialysis module removably attaches to the water treatment module via plumbing connections.

5. The system of claim 3, wherein the dialysis module stacks on top of the water treatment module and the weight of the dialysis module provides a secure coupling between the plumbing connections.

6. The system of claim 1, wherein the dialysis module includes a dialyzer flow balancer that controls flow of dialysate to the dialyzer.

7. The system of claim 1, wherein the user interface module couples to at least one of the other modules via a wired connection.

8. The system of claim 1, wherein the user interface module couples to at least one of the other modules via a wireless connection.

9. The system of claim 1, further comprising a sanitation module adapted to provide a sanitizing solution to the other modules.

10. The system of claim 1, wherein the dialysis module comprises a dialyzer.

11. A sanitation module for sanitizing a flow stream of a dialysis system, comprising;
a sanitizing solution including a sanitizing agent and a tracer agent, the sanitizing solution adapted for sanitizing the flow stream of the dialysis system; and
a conductivity sensor within the flow stream, the tracer adapted to provide the detection by the conductivity sensor of the presence of sanitizing solution in the flow stream of the dialysis system.

12. The sanitation module of claim 11, wherein the tracer agent is sodium chloride.

13. A dialysis system, comprising:
a water supply system;
a dialysate handling system; and
a sanitation module, the water supply system adapted to supply filtered water to the dialysate handling system, wherein the dialysate handling system comprises a supply module, a mixer module, a concentrate control module, and a dialysate module, the dialysate module including an acid concentrate module, a bicarbonate concentrate module, and a sodium chloride concentrate module, the dialysate handling system adapted to prepare the dialysate to a predetermined chemistry and supply the dialysate to a dialyzer, the supply module adapted to supply a diluted acid solution to the supply module, the bicarbonate concentrate module adapted to supply bicarbonate to the mixer module, the sodium chloride concentrate module of the dialysate module adapted to provide a sodium chloride solution to the mixer module, the mixer module adapted to supply dialysate to the dialyzer, a pH sensor provided in the flow stream after the supply module adapted to communicate pH data to the concentrate control module, the concentrate control module adapted to control the acid concentrate module so as to adjust the pH of the solution exiting the supply module to a predetermined value, an alkalinity sensor provided in the flow stream within the mixer module adapted to communicate total alkalinity to the concentrate control module, the concentrate control module adapted to control the bicarbonate concentrate module so as to adjust the total alkalinity of the solution exiting the mixer module to a predetermined value, a conductivity sensor provided after the mixer module adapted to communicate sodium chloride concentration to the concentrate control module, the concentrate control module adapted to control the sodium chloride concentrate module so as to adjust the sodium chloride concentration of the solution exiting the mixer module to a predetermined value, the sanitation module including a sanitizing solution, the sanitizing solution comprising a sanitizing agent and a tracer agent, the sanitation module adapted to supply the sanitizing solution to the mixer module, the sanitizing agent adapted to sanitize the flow stream of the dialysis system, the conductivity sensor adapted to detect the presence of the tracer agent during a sanitizing and rinse process.

14. The dialysis system of claim 13, wherein the tracer agent is sodium chloride.

15. The dialysis system of claim 13, wherein the sanitizing agent comprises sodium hypochlorite.

16. A method for sanitizing a flow stream of a dialyzer system comprising:
providing a predetermined sanitizing solution comprising a sanitizing agent and a tracer agent into a respective flow stream for a predetermined amount of time suitable for sanitizing the flow stream;
providing purified water to the respective flow stream for a predetermined amount of time suitable for flushing out the sanitizing solution from the respective flow stream;
using an electrical conductivity sensor in the flow stream for detecting the presence of the tracer agent, the duration of providing the sanitizing solution predetermined wherein the electrical conductivity sensor detects a predetermined concentration of the tracer agent in the flow stream signifying that sanitizing solution of sufficient concentration is present in the respective flow stream; and
using the electrical conductivity sensor in the flow stream for detecting the presence of the tracer agent, the duration of providing the purified water predetermined wherein the electrical conductivity sensor detects a predetermined concentration of the tracer agent in the flow stream signifying that sanitizing solution is sufficiently rinsed from the respective flow stream.

17. The method of claim 16, wherein providing a predetermined sanitizing solution comprising a sanitizing agent and a tracer agent comprises providing a predetermined sanitizing solution comprising sodium hypochlorite and sodium chloride.
